(19) United States
(12) Patent Application Publication
(10) Pub. No.: US 2021/0030986 A1
(43) Pub. Date: Feb. 4, 2021

(54) NASOPHARYNGEAL AIRWAY WITH CAPNOGRAPHY

(71) Applicant: Donald Reightler, Brunswick, MD (US)

(72) Inventor: Donald Reightler, Brunswick, MD (US)

(21) Appl. No.: 16/939,362

(22) Filed: Jul. 27, 2020

Related U.S. Application Data
(60) Provisional application No. 62/879,791, filed on Jul. 29, 2019.

(51) Int. Cl. A61M 16/04; A61M 16/10; A61B 5/083; A61B 5/00

(52) U.S. Cl. CPC A61M 16/0461; A61M 16/1005; A61M 16/104; A61M 16/0816; A61B 5/742; A61M 2016/103; A61B 5/0836

(57) ABSTRACT

The nasopharyngeal airway is a modified airway adjunct designed to facilitate proper ventilation when connected to an oxygenation system. The presently claimed invention ensures healthcare providers are able to obtain real-time information based on a patient's ventilation and breathing patterns and maximizes supplemental oxygen delivered to a patient, allowing him or her to breathe comfortably while connected to a breathing system. The nasopharyngeal airway with capnography prevents over-inflation of a patient's lungs, which can cause intense discomfort and further exacerbate medical issues, while also providing improved monitoring of a patient's breathing, thereby ensuring the patient is able to breathe properly and maintain the required oxygen levels.

### CROSS-REFERENCE TO RELATED APPLICATION

The present application is related to and claims priority from prior provisional application Ser. No. 62/879,791, filed Jul. 29, 2019 the entire application of which is incorporated herein by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of assistive breathing devices. More specifically, the invention is a nasopharyngeal airway (NPA) with capnography.

### 2. Description of the Related Art

Assistive breathing systems are designed to deliver oxygen to airways. People unable to maintain an open airway for a period of time may need assistance breathing. When a patient is unconscious, their jaw muscles relax, making it possible for the tongue to fall back into the throat and obstruct the patient's airway. Consequently, it is often necessary to use a NPA, a tube inserted into the nasal passageway, to ensure the airway remains open. Such NPAs usually have a flared end at the end of the tube. When inserted, the flared end rests against the nostril outside the nose, preventing the tube from being lost or misplaced inside the patient's nose.

The diameter of NPAs are measured on the French catheter scale or in millimeters (mm), and standardly available sizes include 6.5 mm, 7.0 mm, 7.5 mm, 8.0 mm, and 8.5 mm. The correct length for each patient is selected by measuring the NPA against the patient and selecting the length that reaches from the patient's nostril to his or her earlobe or the angle of his or her jaw.

Capnography is the monitoring of the concentration, also known as the partial pressure, of carbon dioxide (CO₂) in a patient's respiratory gases. Capnography is mainly used to monitor a patient's breathing while under anesthesia or in intensive care. The concentration of CO₂ is generally shown in a graph of expiratory CO₂, as measured in millimeters of mercury (mmHg) over time, as expired volume, or, when a rebreathing system is being used, the inspired CO₂ may be graphed instead. By monitoring the partial pressure of CO₂ inhaled and exhaled, the CO₂ partial pressure in the arterial blood can be indirectly monitored, which is particularly important when the patient has a nasopharyngeal airway in. When the measurement is taken at the end of an exhale, the measurement is referred to as “end-tidal” CO₂ (ETCO₂).

It is particularly important to monitor patient's breathing when they are having assistance breathing. Without knowledge of the patient's CO₂ partial pressure, it is difficult for doctors and nurses to maintain proper pressure levels, and the risk of over inflation of the patient's lungs increases. Over inflation can lead to incorrect diagnosis of other medical problems, which could result in further medical issues.

Ideally, a nasopharyngeal airway with capnography should provide a way to keep a patient's airway open so he or she can breath, while monitoring the patient's breathing and, yet would operate reliably and be manufactured at a modest expense. Thus, a need exists for a reliable nasopharyngeal airway with capnography to avoid the above-mentioned problems.

### BRIEF SUMMARY OF THE INVENTION

In view of the foregoing disadvantages inherent in the known assistive breathing devices art, the present invention provides a novel NPA with capnography. The general purpose of the present invention, which will be described subsequently in greater detail is to provide a nasopharyngeal airway with capnography in order to both assist a patient in breathing while also monitoring his or her breathing at the same time.

The NPA is a modified airway adjunct designed to facilitate proper ventilation when connected to an oxygenation system. The presently claimed invention ensures healthcare providers are able to obtain real-time information based on a patient's ventilation and breathing patterns and maximizes supplemental oxygen delivered to a patient, allowing him or her to breathe comfortably while connected to a breathing system. The nasopharyngeal airway with capnography prevents over-inflation of a patient's lungs, which can cause intense discomfort and further exacerbate medical issues, while also providing improved monitoring of a patient's breathing, thereby ensuring the patient is able to breathe properly and maintain the required oxygen levels.

The present invention holds significant improvements and serves as a NPA with capnography. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention that are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures that accompany the written portion of this specification illustrate embodiments and method(s) of use for the present invention, a nasopharyngeal airway with capnography, constructed and operative according to the teachings of the present invention.

FIG. 1 is a perspective view illustrating a patient breathing comfortably in bed while being monitored, according to an embodiment of the presently claimed invention.

FIG. 2 shows a perspective view illustrating a waveform produced via the breathing system, according to an embodiment of the presently claimed invention.

FIG. 3 is a close-up, see-through view illustrating a NPA with a built-in ETCO₂ monitoring tube, according to an embodiment of the presently claimed invention.

FIG. 4 is an alternate perspective view illustrating a NPA with a built-in ETCO₂ monitoring tube, according to an embodiment of the presently claimed invention.

FIG. 5 is a close-up view illustrating the ETCO₂ tube connector end, according to an embodiment of the presently claimed invention.

The various embodiments of the present invention will hereinafter be described in conjunction with the appended drawings, wherein like designations denote like elements.

### DETAILED DESCRIPTION

As discussed above, embodiments of the present invention relate to a NPA with capnography and more particularly to a NPA as used to improve the patient's breathing and monitoring of the patient's breathing when the patient is connected to an oxygenation system.

Generally speaking, a NPA is an improved airway adjunct used to facilitate proper breathing when connected to an oxygenation system. The system supplies healthcare providers with real-time information based on patient oxygenation levels. The appearance resembles a nasopharyngeal airway with a capnography adapter. The system is inserted into a patient to help facilitate proper breathing. Waveforms are produced on the monitor, enabling nurses, doctors, etc., to maintain constant and effective monitoring of the patient.

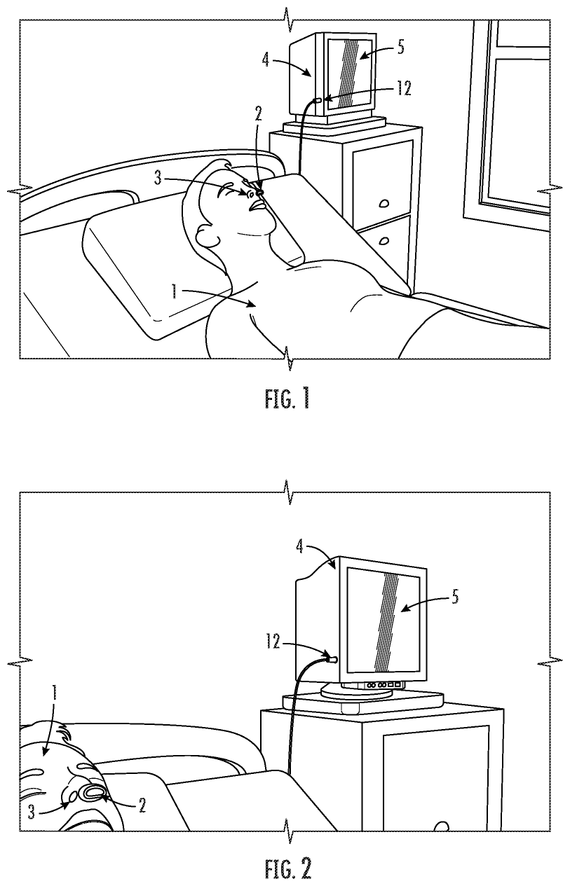

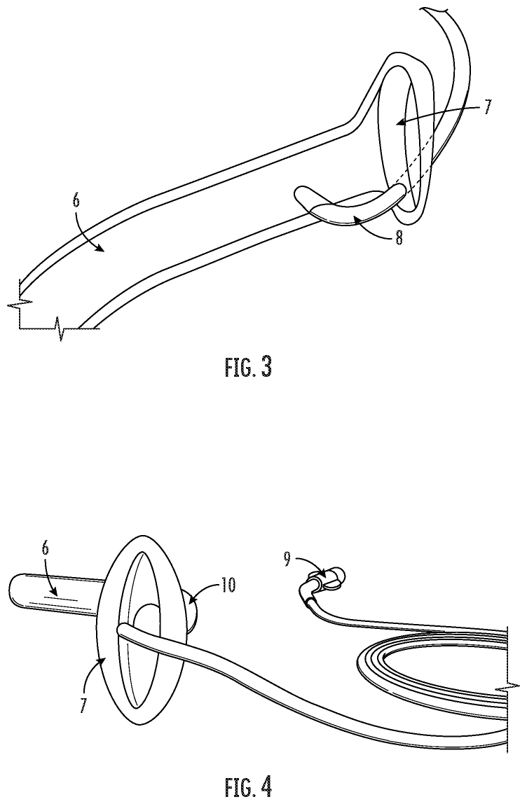

Referring to the drawings by numerals of reference there is shown in FIGS. 1-2, a patient 1 has the NPA 2 inserted through a nostril 3. The NPA 2 is connected to a monitoring device 4. The monitoring device 4 detects a waveform 5 produced by the patient's breaths, thereby giving an indication of the patient's breathing pattern. The monitoring device 4 is a programmable device programmed to adjust the amount of supplemental oxygen supplied to the patient 1 through the NPA 2 based on the patient's ETCO₂.

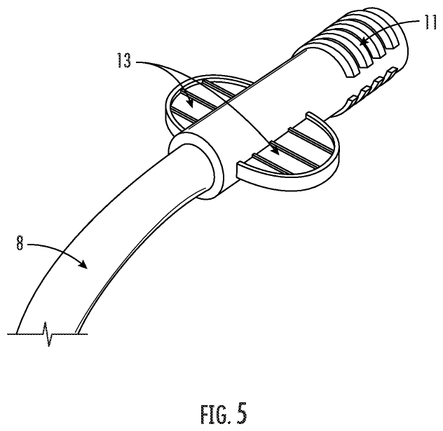

Referring now to FIGS. 3-4, the NPA 2 has a main tube 6 with a flared end 7 to prevent the main tube 6 from sliding too far into the patient's nostril 3. A monitoring and oxygenation tube 8 is connected inside the main tube 6, exiting through the opening of the flared end 7. The monitoring and oxygenation tube 8 connects to the monitoring device 4 via a connector end 9, which is programmed to use the airflow through the monitoring tube 8 to measure the patient's ETCO₂. The main tube 6 may include a bend 10 to better follow a patient's nasal passageway. The monitoring device 4 adjusts the percentage oxygen being supplied back to the patient 1 through the monitoring and oxygenation tube 8 based on the measured ETCO₂, thereby ensuring that the patient's breathing and oxygen level remains stable.

The main body 6 is a pliable, bent cylinder constructed using soft plastic or rubber. The size of the main body 6 may be altered to accommodate specific users' needs and preferences, but generally a tube with a 6 to 7 millimeter outer diameter is used for adult females and a tube with a 7 to 8 millimeter outer diameter is used for adult males.

Referring now to FIG. 5, the connector end 9 is located at the opposite end of the monitoring and oxygenation tube 8 from the main body 6. The connector end 9 has ridges 11 to help hold the connector end 9 in the port 12 on the monitoring device 4. The connector end 9 also has two opposing tabs 13 to provide a grip for removing the connector end 9 from the port 12.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A nasopharyngeal airway with capnography comprising:
a. a main tube having a first end with a diameter equal to that of the tube, and a second flared end with a diameter greater than that of the main tube;
b. a monitoring and oxygenation tube operably connected inside the main tube at a first end so that oxygen may be supplied to the main tube via the monitoring and oxygenation tube and a patient's breath may be monitored by a monitoring device by the monitoring and oxygenation tube;
c. a connection end having
(i) one or more ridges around a circumference of a port end of the connection end; and
(ii) two parallel grip tabs set opposing each other around a circumference of a tube end of the connection end,
wherein the connection end is operably connected at the tube end to a second end of the monitoring and oxygenation tube so that it can be removably plugged into a port on the monitoring device at the port end, thereby connecting the monitoring and oxygenation tube to the monitoring device.

2. The nasopharyngeal airway with capnography according to claim 1, wherein the main tube comprises a soft plastic material or a rubber material.

3. The nasopharyngeal airway with capnography according to claim 1, wherein the main tube is a flexible cylinder with a bend therein to follow a shape of a nasal passageway.

4. The nasopharyngeal airway with capnography according to claim 1, wherein the main tube has an outer diameter of 6 to 7 millimeters.

5. The nasopharyngeal airway with capnography according to claim 1, wherein the main tube has an outer diameter of 7 to 8 millimeters.

6. A nasopharyngeal airway with capnography comprising:
a. a main tube having a first end with a diameter equal to that of the tube, and a second flared end with a diameter greater than that of the main tube,
wherein the main tube is a flexible cylinder formed of a soft plastic material or a rubber material, and
wherein the main tube has a bend therein to follow a shape of a nasal passageway;
b. a monitoring and oxygenation tube operably connected inside the main tube at a first end so that oxygen may be supplied to the main tube via the monitoring and oxygenation tube and a patient's breath may be monitored by a monitoring device by the monitoring and oxygenation tube;
c. a connection end having
(i) one or more ridges around a circumference of a port end of the connection end; and
(ii) two parallel grip tabs set opposing each other around a circumference of a tube end of the connection end,
wherein the connection end is operably connected at the tube end to a second end of the monitoring and oxygenation tube so that it can be removably plugged into a port on the monitoring device at the port end, thereby connecting the monitoring and oxygenation tube to the monitoring device.

7. The nasopharyngeal airway with capnography according to claim 6, wherein the main tube has an outer diameter of 6 to 7 millimeters.

8. The nasopharyngeal airway with capnography according to claim 6, wherein the main tube has an outer diameter of 7 to 8 millimeters.

9. A nasopharyngeal airway system with capnography comprising:
a. a programmable capnography monitoring device with
i. an intake port;
ii. a display monitor;
iii. a memory storage device; and
iv. a programmable processor,
wherein the programmable capnography device is programmed to detect a patient's end-tidal CO₂ via an input connected to the intake port, display a graphical representation of the patient's end-tidal CO₂ on the display monitor, and adjust a flow of oxygen to the patient in response to the patient's end tidal CO₂;
b. a main tube having a first end with a diameter equal to that of the tube, and a second flared end with a diameter greater than that of the main tube;
c. a monitoring and oxygenation tube operably connected inside the main tube at a first end so that oxygen may be supplied to the main tube via the monitoring and oxygenation tube and a patient's breath may be detected by the by programmable monitoring device via the monitoring and oxygenation tube;
d. a connection end having
(i) one or more ridges around a circumference of a port end of the connection end; and
(ii) two parallel grip tabs set opposing each other around a circumference of a tube end of the connection end,
wherein the connection end is operably connected at the tube end to a second end of the monitoring and oxygenation tube so that it can be removably plugged into the intake port on the programmable monitoring device at the port end, thereby connecting the monitoring and oxygenation tube to the monitoring device.
